(19) United States
(12) Patent Application Publication
(10) Pub. No.: US 2019/0019571 A1
(43) Pub. Date: Jan. 17, 2019

(54) Method, System and Computer Program Product for Evaluation of Insulin Sensitivity, Insulin/Carbohydrate Ratio, and Insulin Correction Factors in Diabetes from Self-Monitoring Data

(71) Applicant: University of Virginia Patent Foundation, Charlottesville, VA (US)

(72) Inventors: Marc D. Breton, Charlottesville, VA (US); Boris P. Kovatchev, Charlottesville, VA (US)

(21) Appl. No.: 16/126,879

(22) Filed: Sep. 10, 2018

Related U.S. Application Data
(63) Continuation of application No. 12/665,149, filed on Dec. 17, 2009, now abandoned.
(60) Provisional application No. 60/958,767, filed on Jul. 9, 2007.

(51) Int. Cl. G16H 10/40; G16H 50/30; G06F 19/00; G16H 50/50; G16H 40/63

(52) U.S. Cl. CPC G16H 10/40; G16H 50/30; G16H 40/63; G16H 50/50; G06F 19/00

(57) ABSTRACT

A method, system and computer program product for evaluating or determining a user's insulin sensitivity (SI). An initial step or module may include acquiring SMBG readings from a predetermined period. Another step or module may include computing an estimate of insulin sensitivity (SI) from the SMBG readings. Another step or module may include using the estimate of SI to compute individualized carbohydrate ratio. Additionally, another step or module may include using the estimate of SI to compute individualized correction factor. The computation of the two components of an insulin dose calculator, carbohydrate ratio and correction factor, uses this estimate, which allows the tailoring of carbohydrate ratio and correction factor to the present state of the person.

### RELATED APPLICATIONS

The present invention claims priority from U.S. Provisional Application Ser. No. 60/958,767, filed Jun. 9, 2007, entitled “Method, System and Computer Program Product for Evaluation of Insulin Sensitivity, Insulin/Carbohydrate Ratio, and Insulin Correction Factors in Diabetes from Self-Monitoring Data;” the disclosure of which is hereby incorporated by reference herein in its entirety.

### GOVERNMENT SUPPORT

Work described herein was supported by Federal Grant No. NIH R01DK051562, awarded by the National Institutes of Health. The United States Government has certain rights in this invention.

### BACKGROUND OF THE INVENTION

Insulin Resistance and Insulin Sensitivity in Diabetes:

Diabetes is a complex of disorders, characterized by a common final element of hyperglycemia, that arise from, and are determined in their progress by mechanisms acting at all levels of bio-system organization—from molecular to human behavior. Diabetes mellitus has two major types: Type 1 (T1DM) caused by autoimmune destruction of insulin producing pancreatic beta-cells, and Type 2 (T2DM), caused by defective insulin action (insulin resistance) combined with progressive loss of insulin secretion. Over 20 million people arc currently afflicted by diabetes in the US, with epidemic increases now occurring. The risks and costs of diabetes (over $100 billion/yr) come from its chronic complications in 4 major areas: retinal disease which is the leading cause of adult blindness, renal disease representing half of all kidney failures, neuropathy which predisposes to over 82,000 amputations each year, and cardiovascular disease (CVD), which is 2-4 times more common than in those without diabetes. Cardiovascular disease in diabetes is also more morbid, more lethal and less benefited by modern interventions such as bypass surgery or stents. Thus, the ability of insulin to stimulate glucose metabolism is of fundamental importance in the development and clinical course of diabetes (21, 24, 32). The cluster of changes associated with insulin resistance has been said to comprise syndrome X (21), and all of the manifestations of syndrome X have been shown to increase risk of coronary heart disease. Thus, it is concluded that: “insulin resistance and its associated abnormalities are of utmost importance in the pathogenesis of diabetes, particularly T2DM, hypertension, and coronary heart disease” (32).

A Note on Terminology:

the state of insulin resistance, in which a given amount of insulin produces a less-than-expected effect glucose metabolism, has been known for over 55 years (29). The syndromes of insulin resistance include obesity, glucose intolerance, diabetes, syndrome X, etc. (21, 32). Insulin sensitivity refers to the sensitivity of glucose clearance to plasma insulin variations. Several indexes have been published; the two most used are the clamp insulin sensitivity SI_{(DF)} defined by DeFronzo (18) as the ratio of glucose injection and insulin concentration, and SI_{(BC)} mathematically derived by Bergman and Cobelli from the minimal model of glucose regulation (4). SI_{(DF)} and SI_{(BC)} are highly correlated; the difference between the two is generally in the method of data collection.

In a non-limiting and exemplary approach of the present invention, we use SI as an index of insulin sensitivity, which is derived using the DeFronzo method, unless otherwise specified.

Assessment of Insulin Sensitivity:

Assessment of insulin sensitivity can be done in several ways, but two major protocols have been favored in the past 3 decades: the hyperinsulemic euglycemic clamp and the glucose tolerance test (intravenous or oral, IVGTT or OGTT). The first method is based on the work by DeFronzo et al. (18), which estimates SI as the ratio of the average glucose injection during the last 30 minutes of the protocol divided by the plasma insulin concentration (constant because clamped). It is widely used, referred to in more than 2,200 publications, and generally accepted as a gold standard. The second method uses the glucose-insulin dynamics mathematically characterized by Bergman and Cobelli's now classic Minimal Model (4) and by a number of subsequent studies (3, 6, 7, 11, 31). A recent count showed that the Minimal Model had been used in >600 publications (12). A newer c-peptide minimal model allowed for a more precise evaluation of β-cell function (34, 35, 36). Further research showed that oral glucose tolerance test could be used as well (9, 10, 13, 14, 15). The oral models have been extensively validated in the nondiabetic population, but more work is needed to assess their domain of validity in the diabetes, albeit first results are promising (1). The Minimal Model (2) allows estimating SI_{(BC)} and insulin action (X) from oral or intravenous tests. Usually the model is numerically identified by nonlinear least squares or maximum likelihood.

Disposition Index (DI):

In pre-diabetes, insulin resistance is compensated by increased insulin secretion from the β-cell. Until this compensation fails, ncar-normal glucose tolerance is maintained. If diminished, β-cell responsivity could lead to the development of T2DM. It was shown that in health the relationship between insulin sensitivity and β-cell function, as estimated from the Minimal Model, is hyperbolic, i.e. insulin sensitivity X β-cell function equals a constant (5, 25). FIG. 1 represents this hyperbolic relationship, which indicates normal glucose tolerance (sold line in FIG. 1). For example, state 1 represents normal insulin sensitivity and normal β-cell response, while in state 2 insulin resistance is increased, but the β-cells compensate with increased output. However, if insulin sensitivity decreases and the β-cells can no longer keep up, the hyperbolic relationship is no longer preserved (FIG. 1, dashed line), even if the β-cell function is normal (state 3). The DI has been well documented as a powerful determinant of T2DM (19, 22, 23, 24, 39). In particular, decreased acute β-cell response during the first 8-10 min of glucose infusion (26), has been documented in subjects with diabetes and impaired glucose tolerance, as well as among first-degree relatives of people with T2DM (22).

It is important to note that insulin sensitivity (and therefore DI) is not fixed within a person—these indices change over time and with various modes of treatment. The SI (defined by either formula) is particularly vulnerable to the effects of physical activity, which can increase insulin sensitivity for hours after exercise (30, 33, 38). In general, muscle contraction increases total blood flow to muscle (37) and recruits capillaries (17), thereby increasing the uptake of glucose. Further, insulin sensitivity has natural circadian cycles, e.g. insulin resistance appears to be highest in morning, particularly in T2DM, (8, 28).

Because all metabolic parameters change over time, it follows that a single determination of these parameters is not sufficient for optimizing the treatment regiment of a person with diabetes. This is particularly true for insulin sensitivity because it rapidly changes with the time of day and with the activities of a person.

Therefore methods and systems for tracking the changes in insulin sensitivity are needed for the day-to-day optimization of diabetes control. However, the classic methods of estimation of SI based on euglycemic clamp or on the Minimal Model require invasive hospital-based interventions, with frequent blood sampling for insulin and glucose. Because such invasive procedures cannot be performed frequently on an individual, it is important to find correlates of insulin sensitivity and other metabolic parameters that can be derived from readily available data collected in a person's natural environment, such as self-monitoring blood glucose data (SMBG).

Bolus Calculator:

Insulin boluscs arc traditionally calculated in two phases: First, the amount of insulin is computed that is needed by a person to compensate for the carbohydrate content of an incoming meal. This is done by estimating the amount of carbohydrates to be ingested and multiplying by each person's insulin/carbohydrate ratio. Second, the distance between actual blood glucose (BG) concentration and individual target level is calculated and the amount of insulin to reach target the target is computed. This is done by multiplying the (BG−target) difference by individual insulin correction factor.

A good assessment of each person's carbohydrate ratio and correction factor is critical for the optimal control of diabetes. At this time, such an assessment based on individual evaluation of changing insulin sensitivity, is not available. A key to such an assessment is a proven estimate of SI derived from readily available self-monitoring data.

An aspect of an approach of the present invention focuses on insulin sensitivity (SI)—the most important factor needed for optimal diabetes control. SI is relevant to both T1DM and T2DM, both in terms of assessing the progression of the disease and in terms of maintaining optimal daily regiment. In particular, SI can be used as a base for determining optimal insulin dose and timing of insulin injection. Consequently, an aspect of various embodiments of the present invention provides, among other things, two practically applicable methods assisting with the individual adjustments of insulin/carbohydrate ratio and insulin correction factors.

An aspect of the methods, systems, and computer program products presented in this invention may use routine SMBG data, combined with easily accessible personal parameters. The method and system assessing individual SI is validated by comparison of its results against reference hospital-based assessment of SI computed using DeFronzo's method and data from euglycemic clamp performed on 30 patients with T1DM.

### BRIEF SUMMARY OF INVENTION

An aspect of various embodiments of the present invention provides, but not limited thereto, a method, computer method, system, computer system, computer program product and algorithm for evaluation of insulin sensitivity (SI) from routine self-monitoring blood glucose (SMBG) data. While SI is one of the most important parameters of diabetes, an aspect of this invention also includes methods applying SI to deriving two, person-specific, parameters of diabetes management: (i) carbohydrate ratio used to estimate the amount of insulin needed to compensate for upcoming meal, and (ii) correction factor used to adjust insulin amount so a target glucose level can be reached. The related methods and systems may use routine SMBG data collected over a period of 2-6 weeks (or duration or frequency as desired or required) and is based on our previously developed theory of risk analysis of blood glucose data, in particular on a previously introduced glucose variability measure, the Average Daily Risk Range (ADRR), see PCT International Application No. PCT/US2007/000370, filed Jan. 5, 2007, entitled “Method, System and Computer Program Product for Evaluation of Blood Glucose Variability in Diabetes from Self-Monitoring Data;” of which is hereby incorporated by reference herein in its entirety. For the purposes of this document, SMBG is defined as episodic non-automated determination (typically 3-5 times per day) of blood glucose at diabetic patients' natural environment.

Aspects of Various Embodiments of the Present Invention May Pertain Directly to:
Enhancement of existing SMBG devices by introducing a data interpretation component capable of evaluating insulin sensitivity (or insulin resistance, which is a clinically acceptable term, particularly in Type 2 diabetes). Because insulin sensitivity is difficult to measure, and its assessment is critical to optimizing the treatment of diabetes, this feature can be stand-alone, or combined with the features described below;
Enhancement of existing SMBG devices by introducing a data interpretation component assisting in the calculation of daily insulin requirements, particularly with computing pre-meal carbohydrate ratios and insulin correction factors;
Enhancement by the same features of hand-held devices (personal digital assistants, PDA) intended to assist diabetes management;
Enhancement by the same features of software that retrieves SMBG data—such software is produced by virtually every manufacturer of home BG monitoring devices and is customarily used by patients and health care providers for interpretation of SMBG data. The software can reside on patients personal computers, or be used via Internet portal;
A specific application may be the routine assessment of insulin sensitivity (or insulin resistance) in health-care setting. Such an assessment would include basic measurements (weight, height, insulin dosing) combined with SMBG from a person's memory meter.

Exemplary and Non-Limiting Embodiments of the Invention May Include:
1. A system, method and computer program for computing an estimate of insulin sensitivity (SI) using SMBG readings from a predetermined period, for example 2-6 weeks (or other duration as desired or required) and basic measurements (age, weight, height, insulin units per day);
2. A system, method and computer program using the estimate of SI to compute individualized carbohydrate ratio, which will assist with the adjustment of pre-meal insulin boluses;
3. A system, method and computer program using the estimate of SI to compute individualized correction factor, which will assist with the adjustment of insulin dose needed to achieve certain glucose target;

In one embodiment, the invention provides a computerized method, computer program product and system using running estimates of the SI of a person based on SMBG data collected over a predetermined duration to evaluate changes in insulin requirements.

An aspect of an embodiment of the present invention provides a method for evaluation of insulin sensitivity (SI) of a user from routine self-monitoring blood glucose (SMBG) data. The method comprising: applying the SI to derive at least one component of diabetes management. One of the components may comprise: a carbohydrate ratio used to estimate the amount of insulin needed to compensate for upcoming meal, a correction factor used to adjust insulin amount so a target glucose level can be reached, or both the carbohydrate ratio and the correction factor.

An aspect of an embodiment of the present invention provides a system for evaluating insulin sensitivity (SI) of a user from routine self-monitoring blood glucose (SMBG) data. The system may comprise an acquisition module acquiring plurality of SMBG data points; and a processor. The processor may be programmed to: apply the SI to derive at least one component of diabetes management. At least one of the components may comprise: a carbohydrate ratio used to estimate the amount of insulin needed to compensate for upcoming meal, a correction factor used to adjust insulin amount so a target glucose level can be reached, or both of the carbohydrate ratio and correction factor.

An aspect of an embodiment of the present invention provides a computer program product comprising a computer useable medium having computer program logic for enabling at least one processor in a computer system to evaluate insulin sensitivity (SI) of a user from routine self-monitoring blood glucose (SMBG) data. The computer program logic may comprise: applying the SI to derive at least one component of diabetes management. At least one of the components may comprise: a carbohydrate ratio used to estimate the amount of insulin needed to compensate for upcoming meal, a correction factor used to adjust insulin amount so a target glucose level can be reached, or both of the carbohydrate ratio and the correction factor.

These and other advantages and features of the invention will be made more apparent from the description and the drawings that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings in which:

FIG. 1 graphically illustrates the hyperbolic relationship between insulin sensitivity and β-cell responsivity-disposition index.

FIG. 2 graphically illustrates the dynamics of appearance and clearance of glucose during a meal+insulin bolus;

FIG. 3 graphically illustrates the relationship between SI and its estimates SI1 and SI2;

FIG. 4 graphically illustrates the relationship between carbohydrate ratio computed from SI1 and the “450 rule”—an accepted method for computing carbohydrate ratio;

FIG. 5 graphically illustrates the relationship between correction factor computed from SI1 and the “1800 rule”—an accepted method for computing correction factors.

FIG. 6 provides a simplified flowchart or schematic block diagram of an aspect of an exemplary embodiment of the present invention method, system and computer program product for evaluating or determining a user's insulin sensitivity (SI).

FIG. 7: Functional block diagram for a computer system for implementation of embodiments of the present invention;

FIG. 8: Schematic block diagram for an alternative variation of an embodiment of the present invention relating processors, communications links, and systems;

FIG. 9: Schematic block diagram for another alternative variation of an embodiment of the present invention relating processors, communications links, and systems;

FIG. 10: Schematic block diagram for a third alternative variation of an embodiment of the present invention relating processors, communications links, and systems.

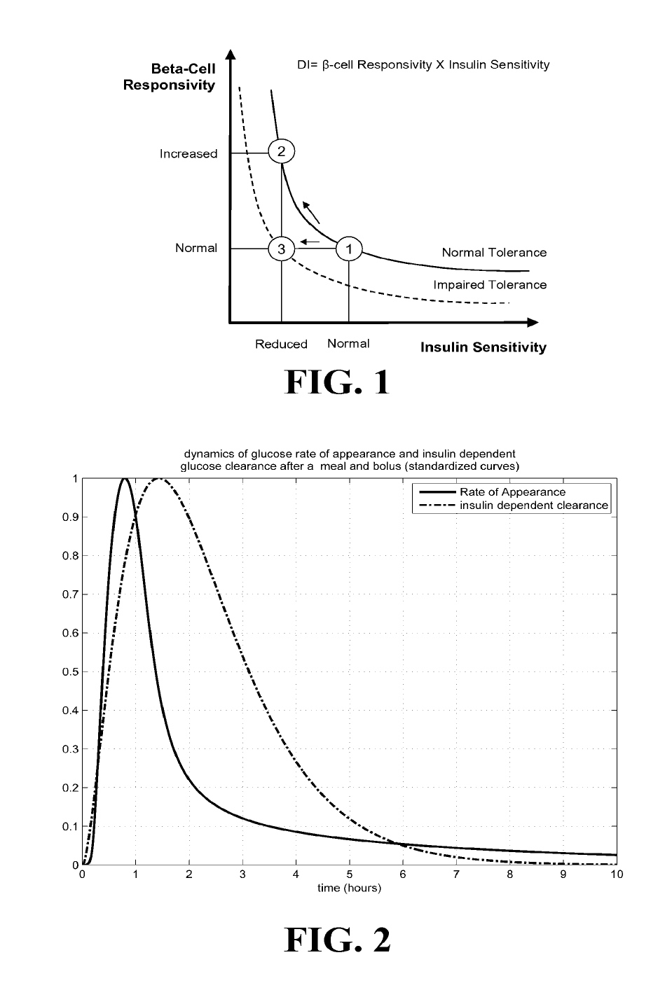

### DETAILED DESCRIPTION OF THE INVENTION

An aspect of an embodiment of the present invention is, but not limited thereto, the estimate of individual insulin sensitivity (SI) derived from personal parameters and SMBG data. The computation of the two components of an insulin dose calculator, carbohydrate ratio and correction factor, uses this estimate, which allows the tailoring of carbohydrate ratio and correction factor to the present state of the person. An aspect of the present invention method or system is the understanding that steady state glucose concentration is controlled via changes in insulin basal rate, while boluses are used to compensate for glycemic events (e.g. meals).

### Data and Data Pre-Processing:

A first step, for example, of computation of insulin sensitivity estimate includes the retrieval of all SMBG data points collected during the last 2-6 weeks of monitoring (or duration as desired or required). These data are then pre-processed as previously described to compute the average daily risk range (ADRR) for a person for this period in time (see U.S. Ser. No. 11/943,226, filed Nov. 20, 2007, entitled “Systems, Methods and Computer Program Codes for Recognition of Patterns of Hyperglycemia and Hypoglycemia, Increased Glucose Variability, and Ineffective Self-Monitoring in Diabetes,” and recently published (27) algorithm, of which are hereby incorporated by reference herein in their entirety). In brief, for a series of SMBG readings x₁, x₂ . . . x_{N} the computation of the ADRR is accomplished by the following formulas:
1. Transform each BG reading into “risk space” using the previously introduced formula: f(BG,a,b)=c. [(ln (BG))^{a}−b)], where the parameters of this function depend on the BG scale and are as follows: If BG is measured in mg/dl, then a=1.084, b=5.381, c=1.509. If BG is measured in mmol/l, then a=1.026, b=1.861 and c=1.794.
2. Compute rl(BG)=r(BG) if f(BG)<0 and 0 otherwise;
   Compute rh(BG)=r(BG) if f(BG)>0 and 0 otherwise.
3. Let x₁¹, x₂¹, . . . xₙ¹ be a series of n¹ SMBG readings taken on Day 1;
   . . .
   Let x₁^{M}, x₂^{M}, . . . xₙ^{M} be a series of n^{M} SMBG readings taken on Day M.
   Where n¹, n², . . . , n^{M}≥3 and the number of days of observation M is between 14 and 42;
4. Compute LRⁱ=max (rl(x₁ⁱ), rl(x₂ⁱ), . . . , rl(xₙⁱ)) and
   HRⁱ=max (rh(x₁ⁱ), rh(x₂ⁱ), . . . , rh(xₙⁱ)) for each day # i; i=1, 2, . . . M.
5. Compute the Average Daily Risk Range as:

$$ADRR = \frac{1}{M} {\sum }_{i = 1}^{M} [ {LR}^{i} + {HR}^{i} ] .$$

A second step, for example, of data collection includes measurement of the following personal parameters:
1. Age and duration of diabetes (these are entered only once);
2. Weight and height to compute body mass index (BMI), recomputed every few months;
3. Typical insulin units per day (or duration as desired or required 0; re-entered whenever the regiment changes significantly.

Estimation of Insulin Sensitivity (SI):

The estimation of SI uses a linear combination of the ADRR and a personal score (SCORE), which is computed by the following computer program:
SCORE=0
if (AGE gt 40) SCORE=SCORE+1
if (DURATION gt 20) SCORE=SCORE+1
if (BMI lt 30) SCORE=SCORE+1
if (INS_KG It 0.5) SCORE=SCORE+1

In other words, one point is added to a basic SCORE of zero for each of the following: Age>40 years, Duration of diabetes>20 years, BMI<30 and insulin units per kilogram weight<0.5 per day. SCORE therefore can range between 0 and 4 for each person, is generally slow-changing, and can change with a person's insulin dose, BMI, or with Age/Duration of diabetes. The estimate of SI is then given by a linear combination of ADRR and SCORE, i.e. by the formula:

SI(EST)=a*ADRR+b*SCORE+c

Several different formulas have been derived using different estimation methods, which are generally equivalent in terms of their SI-predictive ability, and are highly correlates (r>0.99) with each other. The parameters a, b, c of these equivalent formulas are as follows:
a=0.464359; b=5.431937; c=6.613912, which give the estimate:

SI1=0.464359*ADRR+5.431937*SCORE+6.613912,or
a=0.596532; b=6.130669; c=0, which give the estimate:

SI2=0.596532*ADRR+6.130669*SCORE
a=0.430565; b=4.314537; c=10.339625, which give the estimate:

SI3=0.430565*ADRR+4.314537*SCORE+10.339625
a=0.645653; b=5.477073; c=0, which give the estimate:

SI4=0.645653*ADRR+5.477073*SCORE.

Computing Individual Carbohydrate Ratio:

The carbohydrate ratio is used, as previously described, to estimate the amount of insulin needed by a person with diabetes to compensate for ingested glucose. However, it is not possible to exactly match the appearance of glucose in the blood stream with an equal insulin-induced clearance, which leads to the well-known postprandial glucose excursions happening after every meal, even in health. Indeed, as shown graphically in FIG. 2, the dynamics of insulin action in diabetes after a bolus and the rate of appearance of glucose after a meal are quite different. Therefore, optimal diabetes control would mean matching the total amount of glucose entering the system after a meal to the total amount of glucose cleared due to the pre-meal insulin bolus. This is equivalent to equating the integrals of the rate of appearance and the clearance.

Since the insulin sensitivity defined above via clamp data is the amount of additional glucose clearance (in mg·kg⁻¹·min⁻¹) per additional mU/L of insulin, and considering that insulin action follows the largely accepted minimal model dynamics, we can write the total insulin dependent glucose clearance (TIDGC) [mg·kg⁻¹] as:

TIDGC=∫S_{I}·(I−I_{b})=S_{I}∫(I−I_{b})

Wherein I_{b} stands for basal insulin (created by the basal rate alone) and S_{I} stands for insulin sensitivity as defined above.

Now, considering that all infused insulin eventually reaches the blood stream and that plasma insulin clearance is proportional to insulin levels we have:

$$\dot{I} = - N \cdot I + \frac{{rate}_{bolus} + {rate}_{basal}}{V}$$

$$\begin{matrix}\int ( I - {I}_{b} ) = \int \frac{{rate}_{bolus}}{N \cdot V} - \frac{1}{N} \dot{I} \\ = \frac{1}{N \cdot V} \int {rate}_{bolus} - 0 \\ = \frac{bolus}{N \cdot V} \\ = \frac{bolus}{CL}\end{matrix}$$

wherein N is a time constant of insulin diffusion and V a volume of insulin diffusion (neither are not necessarily used in further computation)

The last two equations lead to the formula:

$$TIDGC = {S}_{I} \frac{bolus [ mU ]}{CL}$$

where CL is a subject-specific parameter dependent on insulin clearance and insulin diffusion volume. CL is approximated using field-measurable subject characteristics as follows:

CL=e^{-0.2+0.45·BSA-0.00287·age}

BSA=0.20247·Height[m]^{0.725}·W[kg]^{0.425}

where BSA stands for body surface area.

Equivalently, we can compute the total amount of glucose per kg ingested (TGI):

$$TGI = \frac{1000 \cdot meal amount [ g ]}{weight [ kg ]}$$

Finally, we know that TGI needs to equate TIDGC for an optimal bolus. Therefore:

$$TGI = TIDGC$$

$$1000 \frac{meal}{weight} = {S}_{I} \frac{bolus [ mU ]}{CL}$$

$$\frac{bolus [ U ]}{meal [ g ]} = \frac{CL}{{S}_{I} \cdot weight [ kg ]}$$

$$carb ratio = \frac{CL}{{S}_{I} \cdot weight [ kg ]}$$

Considering SI1 estimates the cumulative sum over 1 hour of glucose utilization per kg of body mass and concentration units (U/L) we need to adjust for diffusion volume of insulin and body weight and divide by 60 (minute value summed over 1 h). The Carbohydrate Ratio is then estimated using the following routine:

CarbRatio=60*V_{I}/SI1=3/SI1

(because V_{I} is fixed at 0.05 L·kg⁻¹ as per literature value of insulin diffusion volume.)

Computing Individual Correction Factor:

The correction factor represents a change in insulin for the purpose of clearing certain amount of glucose from the bloodstream, i.e. for the purpose of bringing BG from its current level to a target level. Therefore the problem can be summarized as equating an additional integral insulin dependent glucose clearance to the observed difference between plasma glucose concentration and targeted glucose concentration:

$$TIDGC = \frac{( BG - {BG}_{target} ) Vol [ dl ]}{weight}$$

$${S}_{I} \frac{bolus [ mU ]}{CL} = \frac{Δ BG \cdot Vol [ dl ]}{weight}$$

$$\frac{bolus [ U ]}{Δ BG [ mg \text{/} dl ]} = 0.001 \times Vol [ dL ] \frac{CL}{{S}_{I} \cdot weight [ kg ]}$$

where ΔBG=(BG−BG_{target}), and Vol stands for glucose diffusion volume (see below) This in turn leads to the formula:

correction factor=0.001×Vol[dl]×carbratio

where the glucose diffusion volume is determined using field-accessible covariates, for example as published in (16): Vol=2.5*weight[kg]

Following this algorithm, the Correction Factor is then estimated using the following routine:

CorrectionFactor=0.001*2.5*Weight[kg]*CarbRatio.

Finally, both ratios assume that all of the subcutaneously injected insulin reaches the central system and acts upon glucose clearance. The validity of this assumption is highly dependent on the type of insulin injected. For common rapid acting insulin it has been shown (20) that about 75% of the injected insulin reach the central system (or other applicable reach factor). Therefore the carbohydrate ratio and correction factor need to be adjusted as follow:

CarbRatio_fast=CarbRatio/0.75

CorrectionFactor_fast=CorrectionFactor/0.75

In general, the values estimated by the method proposed in this invention produce results that need to be adjusted for the type of insulin used. The adjustment coefficients for a number of insulin types and mixtures are given in (20) and range from 0.75 for fast-acting insulin (e.g. regular or Lispro) to 0.3-0.4 for slow-acting insulin (e.g. NPH or lente). For insulin pump users, a fixed adjustment coefficient of 0.75 should be generally acceptable.

Validation of the SI Estimate with Reference Hospital Data:

The estimation of the SMBG-based estimate of SI was validated via comparisons with reference measurement of SI done by the DeFronzo's method using data collected during hyperinsulin clamp study performed in a hospital setting. Thirty adults with T1DM, average age=42.5±12 years, duration of diabetes=21.6±9.4 years, HbA_{1c}=7.4±0.8, 16 males, were hospitalized and their BG was controlled overnight at ˜6 mmol/l. Hyperinsulinemic clamp (1 mU/kg/minute) was initiated in the morning, beginning with 2-hour euglycemia at ˜5.5 mmol/l, followed by 1-hour descent into hypoglycemia with a target level of 2.2 mmol/l. BG was sampled every 5 minutes (Beckman glucose analyzer) to measure SI. The same subjects also performed routine SMBG for 30 days, 4-5 times/day. The ADRR was computed from these SMBG as described above. Demographic and other personal parameters were collected as well.

Table 1 shows the correlation of the clamp-estimated SI with demographic and SMBG-derived parameters. All correlations are in the expected direction, and some notable are in bold.

TABLE 1
Relationship between SI and personal parameters

| | Correlation, p-value | |
|---|---|---|
| Age | 0.32 | (p = 0.08) |
| Duration of Diabetes | 0.32 | (p = 0.08) |
| Body Mass Index (BMI) | −0.33 | (p = 0.08) |
| HbA1c | −0.13 | (n.s.) |
| Insulin units/kg/day | −0.47 | (p = 0.01) |
| Basal Insulin (for pump users, N = 22) | −0.49 | (p = 0.02) |
| Mean BG | −0.04 | (n.s) |
| SD of BG | 0.32 | (p = 0.08) |
| Low BG Index (LBGI) | 0.40 | (p = 0.027) |
| High BG Index (HBGI) | 0.07 | (n.s.) |
| Average Daily Risk Range (ADRR) | 0.57 | (p = 0.001) |

It is evident that ADRR is a most significant predictor of SI, but other parameters can be used to improve this relationship. Thus, we compute SCORE as presented in the previous section. Table 2 presents the distribution of the hospital-measured SI along the levels of SCORE. It is evident that higher SCORE generally corresponds to higher insulin sensitivity:

TABLE 2
Distribution of SI along the levels of SCORE

| | Average SI |
|---|---|
| Score = 0 | 20.9 |
| Score = 1 | 29.3 |
| Score = 2 | 26.9 |
| Score = 3 | 39.0 |
| Score = 4 | 46.6 |

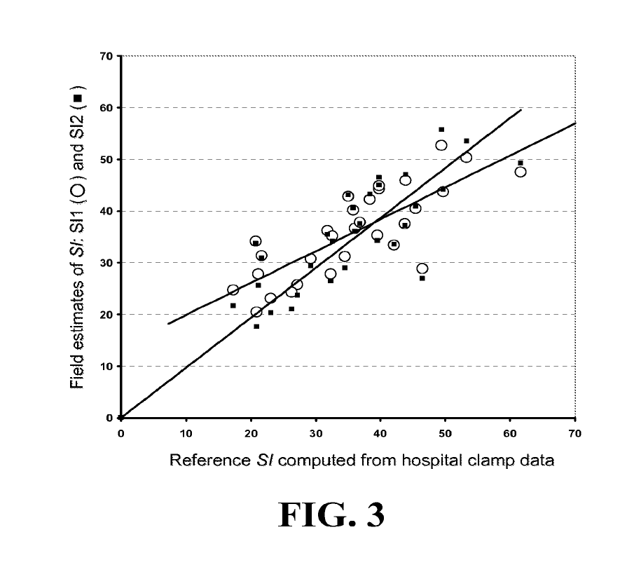

FIG. 3 presents the relationship between the reference SI (x-axis) and its estimates SI1 and SI2 computed by the first two formulas presented above. The correlation between SI with SI1 is r=0.785; with SI2 is r=0.784; with SI3 is r=0.784, and with SI4 is r=0.779 (all p-levels<0.001). Thus, all four estimates provide good approximation of reference insulin sensitivity and any of them can be used as a field data-based approximation of insulin sensitivity.

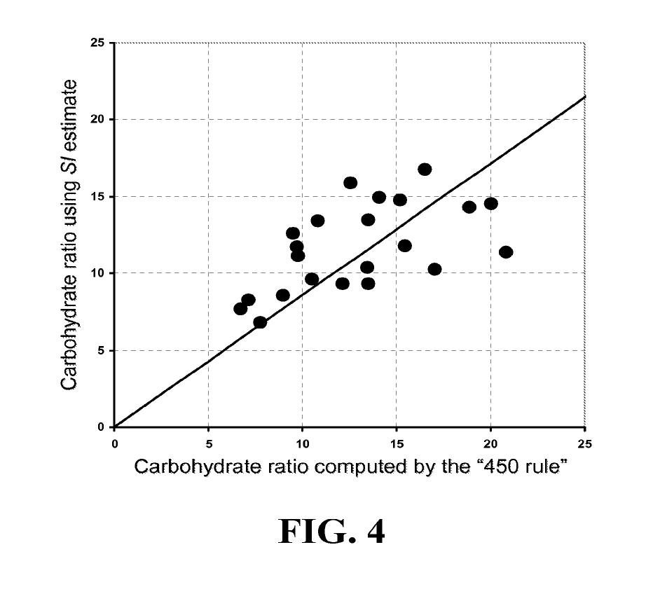

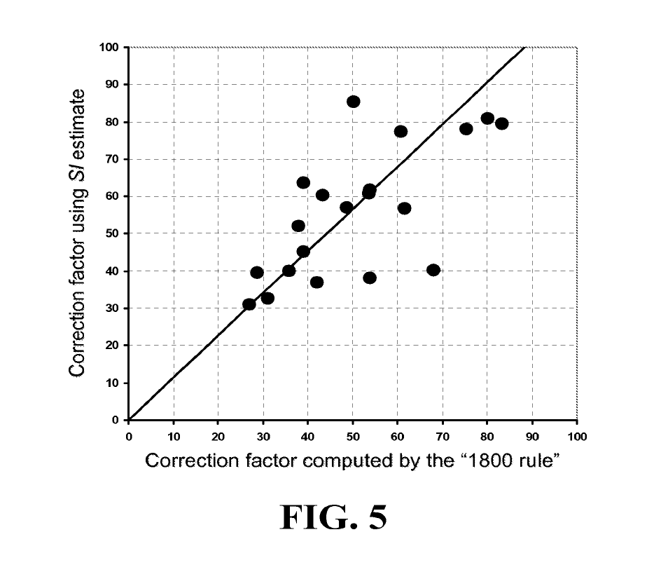

Using total daily insulin from the same field study we can compare our SI-based estimates of carbohydrate ratio and correction factor to the commonly accepted 450 rule and 1800 rule (carb_ratio=dailyinsulin/450, corr_factor=daily_insulin/1800). FIGS. 4 and 5 present scatter plots of the inverse of the SMBG estimated carbohydrates ratio vs. the inverse of the 450 rule calculated ratio. We observe good correlations (˜0.6 for both) and equivalent ranges (˜15 g/i·unit for carb ratio and ˜60 mg/dl per insulin unit for correction factor). Thus the SMBG estimates are comparable to the 450 and 1800 rules, commonly used as a starting point of insulin therapy.

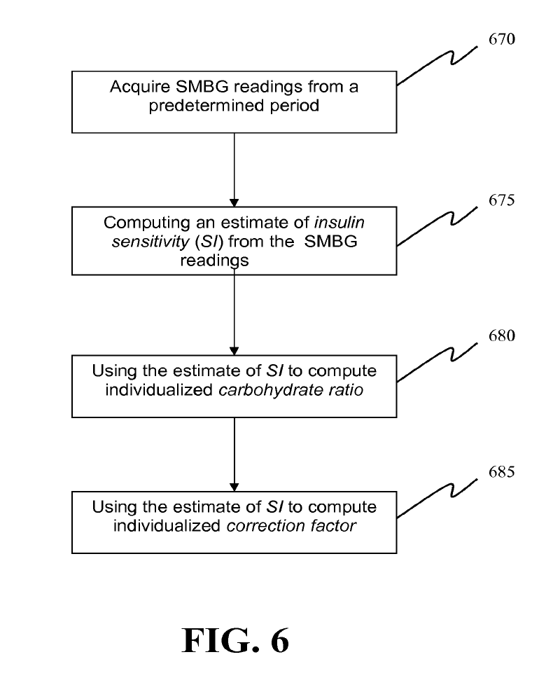

FIG. 6 provides a simplified flowchart or schematic block diagram of an aspect of an exemplary embodiment of the present invention method, system and computer program product for evaluating or determining a user's insulin sensitivity (SI). An initial step or module may include acquiring SMBG readings from a predetermined period 670. Another step or module may include computing an estimate of insulin sensitivity (SI) from the SMBG readings 675. Another step or module may include using the estimate of SI to compute individualized carbohydrate ratio 680. Additionally, another step or module may include using the estimate of SI to compute individualized correction factor 685. The computation of the two components of an insulin dose calculator, carbohydrate ratio and correction factor, uses this estimate, which allows the tailoring of carbohydrate ratio and correction factor to the present state of the person.

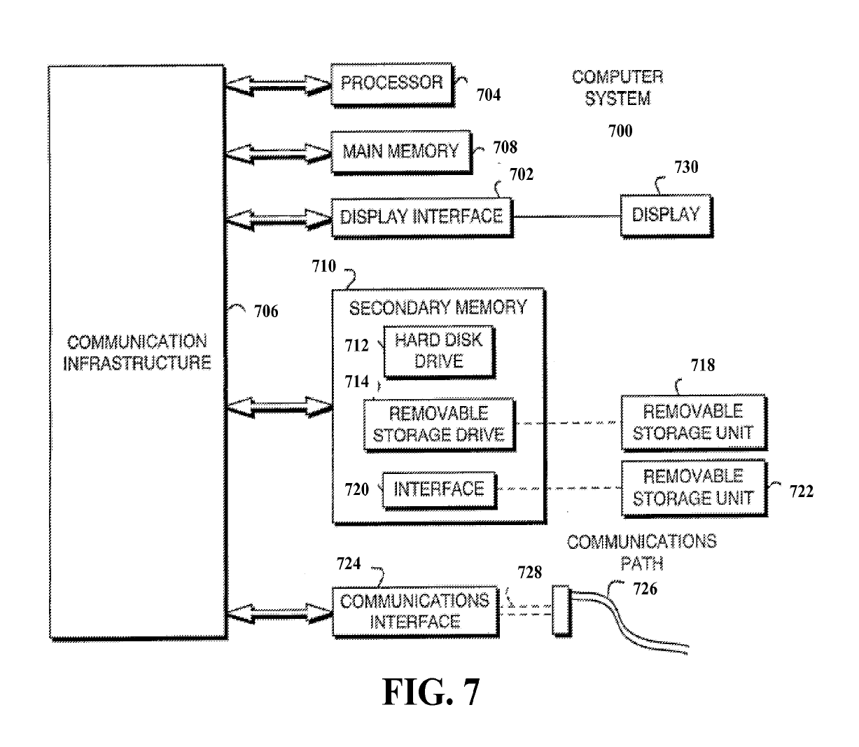

Turning to FIG. 7, FIG. 7 is a functional block diagram for a computer system 700 for implementation of an exemplary embodiment or portion of an embodiment of present invention. For example, a method or system of an embodiment of the present invention may be implemented using hardware, software or a combination thereof and may be implemented in one or more computer systems or other processing systems, such as personal digit assistants (PDAs) equipped with adequate memory and processing capabilities, or directly into blood glucose self-monitoring devices (e.g., SMBG memory meters) equipped with adequate memory and processing capabilities. In an example embodiment, the invention was implemented in software running on a general purpose computer 700 as illustrated in FIG. 7. The computer system 700 may includes one or more processors, such as processor 704. The Processor 704 is connected to a communication infrastructure 706 (e.g., a communications bus, cross-over bar, or network). The computer system 700 may include a display interface 702 that forwards graphics, text, and/or other data from the communication infrastructure 706 (or from a frame buffer not shown) for display on the display unit 730. Display unit 830 may be digital and/or analog.

The computer system 700 may also include a main memory 708, preferably random access memory (RAM), and may also include a secondary memory 710. The secondary memory 710 may include, for example, a hard disk drive 712 and/or a removable storage drive 714, representing a floppy disk drive, a magnetic tape drive, an optical disk drive, a flash memory, etc. The removable storage drive 714 reads from and/or writes to a removable storage unit 718 in a well known manner. Removable storage unit 718, represents a floppy disk, magnetic tape, optical disk, etc. which is read by and written to by removable storage drive 714. As will be appreciated, the removable storage unit 718 includes a computer usable storage medium having stored therein computer software and/or data.

In alternative embodiments, secondary memory 710 may include other means for allowing computer programs or other instructions to be loaded into computer system 700. Such means may include, for example, a removable storage unit 722 and an interface 720. Examples of such removable storage units/interfaces include a program cartridge and cartridge interface (such as that found in video game devices), a removable memory chip (such as a ROM, PROM, EPROM or EEPROM) and associated socket, and other removable storage units 722 and interfaces 720 which allow software and data to be transferred from the removable storage unit 722 to computer system 700.

The computer system 700 may also include a communications interface 724. Communications interface 724 allows software and data to be transferred between computer system 700 and external devices. Examples of communications interface 724 may include a modem, a network interface (such as an Ethernet card), a communications port (e.g., serial or parallel, etc.), a PCMCIA slot and card, a modem, etc. Software and data transferred via communications interface 724 arc in the form of signals 728 which may be electronic, electromagnetic, optical or other signals capable of being received by communications interface 724. Signals 728 are provided to communications interface 724 via a communications path (i.e., channel) 726. Channel 726 (or any other communication means or channel disclosed herein) carries signals 728 and may be implemented using wire or cable, fiber optics, blue tooth, a phone line, a cellular phone link, an RF link, an infrared link, wireless link or connection and other communications channels.

In this document, the terms “computer program medium” and “computer usable medium” are used to generally refer to media or medium such as various software, firmware, disks, drives, removable storage drive 714, a hard disk installed in hard disk drive 712, and signals 728. These computer program products (“computer program medium” and “computer usable medium”) are means for providing software to computer system 700. The computer program product may comprise a computer useable medium having computer program logic thereon. The invention includes such computer program products. The “computer program product” and “computer useable medium” may be any computer readable medium having computer logic thereon.

Computer programs (also called computer control logic or computer program logic) are may be stored in main memory 708 and/or secondary memory 710. Computer programs may also be received via communications interface 724. Such computer programs, when executed, enable computer system 700 to perform the features of the present invention as discussed herein. In particular, the computer programs, when executed, enable processor 704 to perform the functions of the present invention. Accordingly, such computer programs represent controllers of computer system 700.

In an embodiment where the invention is implemented using software, the software may be stored in a computer program product and loaded into computer system 700 using removable storage drive 714, hard drive 712 or communications interface 724. The control logic (software or computer program logic), when executed by the processor 704, causes the processor 704 to perform the functions of the invention as described herein.

In another embodiment, the invention is implemented primarily in hardware using, for example, hardware components such as application specific integrated circuits (ASICs). Implementation of the hardware state machine to perform the functions described herein will be apparent to persons skilled in the relevant art(s).

In yet another embodiment, the invention is implemented using a combination of both hardware and software.

In an example software embodiment of the invention, the methods described above may be implemented in SPSS control language or C++ programming language, but could be implemented in other various programs, computer simulation and computer-aided design, computer simulation environment, MATLAB, or any other software platform or program, windows interface or operating system (or other operating system) or other programs known or available to those skilled in the art.

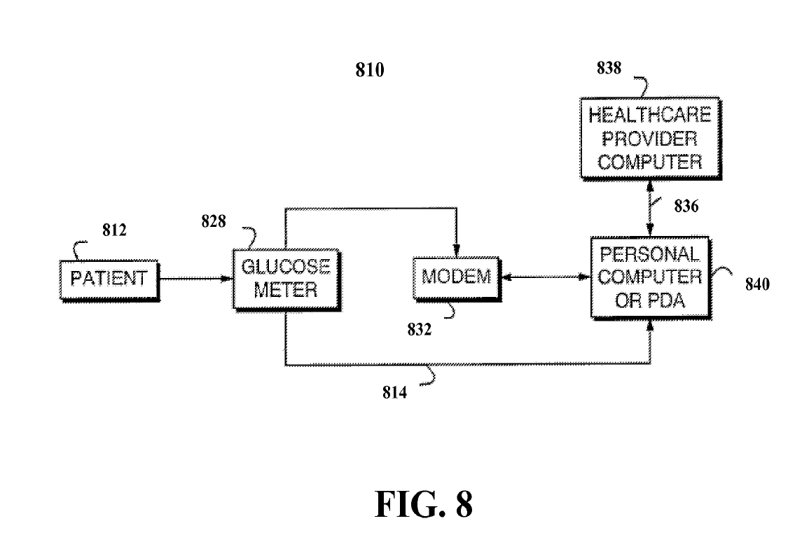

FIGS. 8-10 show block diagrammatic representations of alternative embodiments of the invention. Referring to FIG. 8, there is shown a block diagrammatic representation of the system 810 essentially comprises the glucose meter 828 used by a patient 812 for recording, inter alia, insulin dosage readings and measured blood glucose (“BG”) levels. Data obtained by the glucose meter 828 is preferably transferred through appropriate communication links 814 or data modem 832 to a processor, processing station or chip 840, such as a personal computer, PDA, or cellular telephone, or via appropriate Internet portal. For instance data stored may be stored within the glucose meter 828 and may be directly downloaded into the personal computer 840 through an appropriate interface cable and then transmitted via the Internet to a processing location. An example is the ONE TOUCH monitoring system or meter by LifeScan, Inc. which is compatible with IN TOUCH software which includes an interface cable to download the data to a personal computer. It should be appreciated that the glucose meter 828 and any of the computer processing modules or storage modules may be integral within a single housing or provided in separate housings.

The glucose meter is common in the industry and includes essentially any device that can function as a BG acquisition mechanism. The BG meter or acquisition mechanism, device, tool or system includes various conventional methods directed towards drawing a blood sample (e.g. by fingerprick) for each test, and a determination of the glucose level using an instrument that reads glucose concentrations by electromechanical methods. Recently, various methods for determining the concentration of blood analytes without drawing blood have been developed. For example, U.S. Pat. No. 5,267,152 to Yang et al. (hereby incorporated by reference) describes a noninvasive technique of measuring blood glucose concentration using ncar-IR radiation diffuse-reflection laser spectroscopy. Similar near-IR spectrometric devices are also described in U.S. Pat. No. 5,086,229 to Rosenthal et al. and U.S. Pat. No. 4,975,581 to Robinson et al. (of which are hereby incorporated by reference).

U.S. Pat. No. 5,139,023 to Stanley (hereby incorporated by reference) describes a transdermal blood glucose monitoring apparatus that relies on a permeability enhancer (e.g., a bile salt) to facilitate transdermal movement of glucose along a concentration gradient established between interstitial fluid and a receiving medium. U.S. Pat. No. 5,036,861 to Sembrowich (hereby incorporated by reference) describes a passive glucose monitor that collects perspiration through a skin patch, where a cholinergic agent is used to stimulate perspiration secretion from the ecerine sweat gland. Similar perspiration collection devices are described in U.S. Pat. No. 5,076,273 to Schoendorfer and U.S. Pat. No. 5,140,985 to Schroeder (of which are hereby incorporated by reference).

In addition, U.S. Pat. No. 5,279,543 to Glikfeld (hereby incorporated by reference) describes the use of iontophoresis to noninvasively sample a substance through skin into a receptacle on the skin surface. Glikfeld teaches that this sampling procedure can be coupled with a glucose-specific biosensor or glucose-specific electrodes in order to monitor blood glucose. Moreover, International Publication No. WO 96/00110 to Tamada (hereby incorporated by reference) describes an iotophoretic apparatus for transdermal monitoring of a target substance, wherein an iotophoretic electrode is used to move an analyte into a collection reservoir and a biosensor is used to detect the target analyte present in the reservoir. Finally, U.S. Pat. No. 6,144,869 to Berner (hereby incorporated by reference) describes a sampling system for measuring the concentration of an analyte present.

Further yet, the BG meter or acquisition mechanism may include indwelling catheters and subcutaneous tissue fluid sampling.

The computer, processor or PDA 840 may include the software and hardware necessary to process, analyze and interpret the self-recorded diabetes patient data in accordance with predefined flow sequences and generate an appropriate data interpretation output. The results of the data analysis and interpretation performed upon the stored patient data by the computer 840 may be displayed in the form of a paper report generated through a printer associated with the personal computer 840. Alternatively, the results of the data interpretation procedure may be directly displayed on a video display unit associated with the computer 840. The results additionally may be displayed on a digital or analog display device. The personal computer 840 may transfer data to a healthcare provider computer 838 through a communication network 836. The data transferred through communications network 836 may include the self-recorded diabetes patient data or the results of the data interpretation procedure.

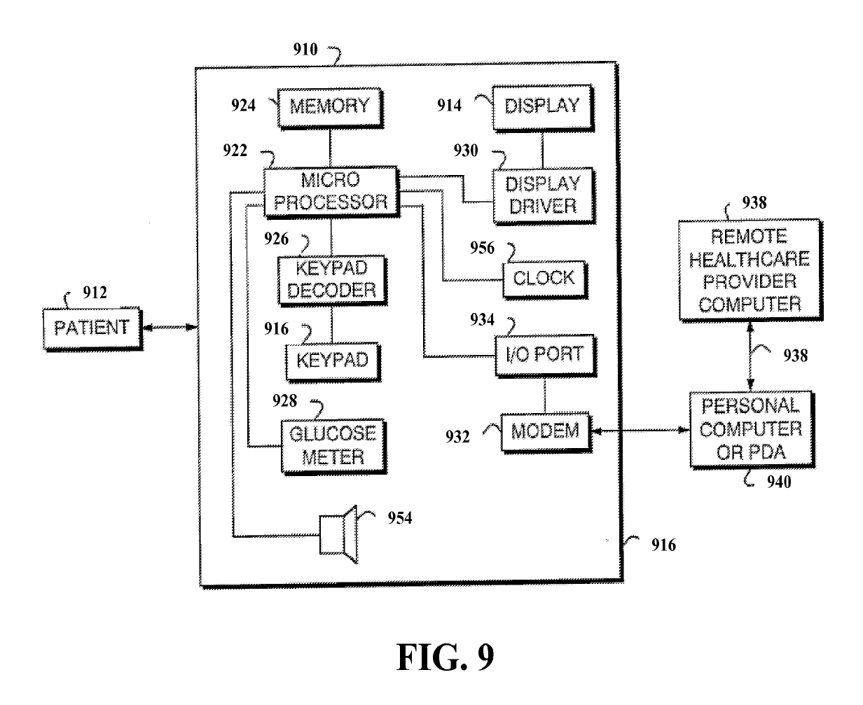

FIG. 9 shows a block diagrammatic representation of an alternative embodiment having a diabetes management system that is a patient-operated apparatus 910 having a housing preferably sufficiently compact to enable apparatus 910 to be hand-held and carried by a patient. A strip guide for receiving a blood glucose test strip (not shown) is located on a surface of housing 916. Test strip receives a blood sample from the patient 912. The apparatus may include a microprocessor 922 and a memory 924 connected to microprocessor 922. Microprocessor 922 is designed to execute a computer program stored in memory 924 to perform the various calculations and control functions as discussed in greater detail above. A keypad 916 may be connected to microprocessor 922 through a standard keypad decoder 926. Display 914 may be connected to microprocessor 922 through a display driver 930. Display 914 may be digital and/or analog. Speaker 954 and a clock 956 also may be connected to microprocessor 922. Speaker 954 operates under the control of microprocessor 922 to emit audible tones alerting the patient to possible future hypoglycemic or hyperglycemic risks. Clock 956 supplies the current date and time to microprocessor 922.

Memory 924 also stores blood glucose values of the patient 912, the insulin dose values, the insulin types, and the parameters used by the microprocessor 922 to calculate future blood glucose values, supplemental insulin doses, and carbohydrate supplements. Each blood glucose value and insulin dose value may be stored in memory 924 with a corresponding date and time. Memory 924 is preferably a non-volatile memory, such as an electrically erasable read only memory (EEPROM).

Apparatus 910 may also include a blood glucose meter 928 connected to microprocessor 922. Glucose meter 928 may be designed to measure blood samples received on blood glucose test strips and to produce blood glucose values from measurements of the blood samples. As mentioned previously, such glucose meters are well known in the art. Glucose meter 928 is preferably of the type which produces digital values which are output directly to microprocessor 922. Alternatively, blood glucose meter 928 may be of the type which produces analog values. In this alternative embodiment, blood glucose meter 928 is connected to microprocessor 922 through an analog to digital converter (not shown).

Apparatus 910 may further include an input/output port 934, preferably a serial port, which is connected to microprocessor 922. Port 934 may be connected to a modem 932 by an interface, preferably a standard RS232 interface. Modem 932 is for establishing a communication link between apparatus 910 and a personal computer 940 or a healthcare provider computer 938 through a communication network 936. Specific techniques for connecting electronic devices through connection cords are well known in the art. Another alternative example is “Bluetooth” technology communication.

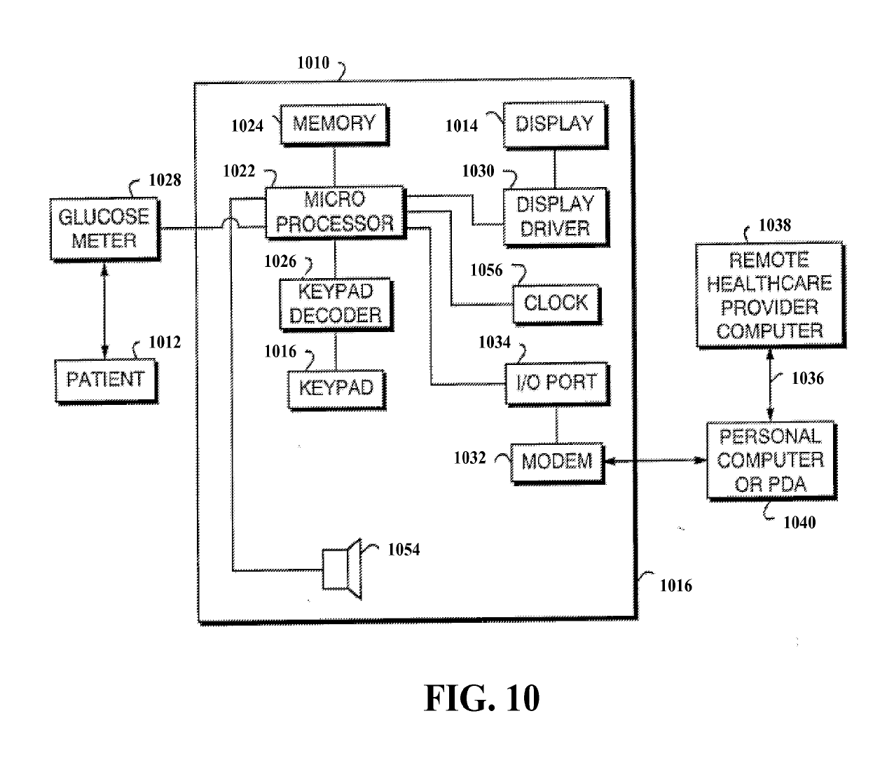

Alternatively, FIG. 10 shows a block diagrammatic representation of an alternative embodiment having a diabetes management system that is a patient-operated apparatus 1010, similar to the apparatus as shown in FIG. 9, having a housing preferably sufficiently compact to enable the apparatus 1010 to be hand-held and carried by a patient. For example, a separate or detachable glucose meter or BG acquisition mechanism/module 1028. There are already self-monitoring devices that are capable of directly computing the algorithms disclosed in this application and displaying the results to the patient without transmitting the data to anything else. Examples of such devices are ULTRA SMART by LifeScan, Inc., Milpitas, Calif. and FREESTYLE TRACKER by Therasense, Alameda, Calif.

Accordingly, the embodiments described herein are capable of being implemented over data communication networks such as the internet, making evaluations, estimates, and information accessible to any processor or computer at any remote location, as depicted in FIGS. 7-10 and/or U.S. Pat. No. 5,851,186 to Wood, of which is hereby incorporated by reference herein. Alternatively, patients located at remote locations may have the BG data transmitted to a central healthcare provider or residence, or a different remote location.

It should be appreciated that any of the components/modules discussed in FIGS. 7-10 may be integrally contained within one or more housings or separated and/or duplicated in different housings.

It should also be appreciated that any of the components/modules present in FIGS. 7-10 may be in direct or indirect communication with any of the other components/modules.

In summary, the various embodiments of the invention propose a data analysis computerized (or non-computerized) method and system for quantifying insulin sensitivity using episodic self-monitoring BG (SMBG) data combined with obtainable individual parameters, such as age and body mass index (BMI).

As an additional advantage, the various embodiments of the invention enhance hand-held devices (e.g. PDAs or any applicable devices or systems) intended to assist diabetes management.

Still yet another advantage, the various embodiments of the invention enhance software that retrieves SMBG data. This software can reside on patients' personal computers, or be used via Internet portal.

Moreover, the various embodiments of the invention may evaluate the effectiveness of various treatments for diabetes (e.g. insulin or variability lowering medications, such as pramlintide and exenatide).

Further still, the various embodiments of the invention may evaluate the effectiveness of new insulin delivery devices (e.g. insulin pumps), or of future closed-loop diabetes control systems.

The methods and systems of the present invention can be used separately, in combination, or in addition to previously described methods, to drive a system of messages delivered by the device to an individual with diabetes, in this case at a time proximal to a patient BG test. A theoretical model of self-regulation behavior asserts that such messages would be effective and would result in improved glycemic control, for example.

In summary, insulin sensitivity (or its inverse, insulin resistance) is one of the most important for treatment of diabetes individual parameter. However, precise estimates of insulin sensitivity from widely available field data are currently not available—the estimation of insulin sensitivity requires lab-based blood testing of glucose and insulin values.

An aspect of an embodiment of the present invention comprises of a method, computer method, system, computer system, device and computer program product for quantifying insulin sensitivity using routine episodic self-monitoring BG (SMBG) data combined with several easily obtainable individual parameters, such as age and body mass index. The methods and systems are based on in part our previously developed theory of risk analysis of BG data; in particular on a recently reported measure of glucose variability—the Average Daily Risk Range (ADRR). The computation of insulin sensitivity has been validated via comparison with data for 30 patients with type 1 diabetes obtained during euglycemic clamp study performed in a hospital setting. The correlation between reference laboratory insulin sensitivity and its estimates from field data was >0.75.

Based on insulin sensitivity estimates, an aspect of the present invention further provides individual tailoring of two most important parameters of diabetes management: insulin/carbohydrate ratio and correction factor. Such adjustments could be recommended by a self-monitoring device with the accumulation of self-monitoring data.

In summary, the computation of individualized insulin/carbohydrate ratio and correction factor is now possible from estimates of individual insulin sensitivity derived from field data. These estimates have also stand-alone value, particularly in type 2 diabetes where insulin resistance is a major factor for assessment and treatment.

Blood glucose self-monitoring devices are the current standard observational practice in diabetes, providing routine SMBG data that serve as the main feedback enabling patients to maintain their glycemic control. An aspect of an embodiment of the present invention provides, but not limited thereto, the following SMBG-related applications:
Provide accurate evaluation of one of the most important parameters of diabetes control—insulin sensitivity (or insulin resistance)—by way of a field test based on routine self-monitoring (SMBG) data;
Provide evaluation of individualized insulin/carbohydrate ratio and correction factor based on individual insulin sensitivity;
Serve as a measure for assessment the effectiveness of medications reducing insulin sensitivity in diabetes (such as metformin); and
Serve as a field assessment of insulin resistance in type 2 diabetes.

Some non-limiting and exemplary advantages attributed with the present invention methods and systems over the existing technologies include: (i) Tracking of changes in insulin sensitivity from readily available routine self-monitoring data; (ii) Individualized assessment of insulin/carbohydrate ratio and correction factor that changes over time with the changes of a person's insulin sensitivity.

It should be appreciated that various aspects of embodiments of the present method, system, devices and computer program product may be implemented with the following methods, systems, devices and computer program products disclosed in the following U.S. Patent Applications, U.S. Patents, and PCT International Patent Applications that are hereby incorporated by reference herein and co-owned with the assignee:

PCT/US2008/067725, entitled “Method, System and Computer Simulation Environment for Testing of Monitoring and Control Strategies in Diabetes,” filed Jun. 20, 2008;

PCT/US2007/085588 not yet published filed Nov. 27, 2007, entitled “Method, System, and Computer Program Product for the Detection of Physical Activity by Changes in Heart Rate, Assessment of Fast Changing Metabolic States, and Applications of Closed and Open Control Loop in Diabetes;”

U.S. Ser. No. 11/943,226, filed Nov. 20, 2007, entitled “Systems, Methods and Computer Program Codes for Recognition of Patterns of Hyperglycemia and Hypoglycemia, Increased Glucose Variability, and Ineffective Self-Monitoring in Diabetes;”

PCT International Application Serial No. PCT/US2005/013792, filed Apr. 21, 2005, entitled “Method, System, and Computer Program Product for Evaluation of the Accuracy of Blood Glucose Monitoring Sensors/Devices;”

U.S. patent application Ser. No. 11/578,831, filed Oct. 18, 2006 entitled “Method, System and Computer Program Product for Evaluating the Accuracy of Blood Glucose Monitoring Sensors/Devices;”

PCT International Application Serial No. PCT/US01/09884, filed Mar. 29, 2001, entitled “Method, System, and Computer Program Product for Evaluation of Glycemic Control in Diabetes Self-Monitoring Data;”

U.S. Pat. No. 7,025,425 B2 issued Apr. 11, 2006, entitled “Method, System, and Computer Program Product for the Evaluation of Glycemic Control in Diabetes from Self-Monitoring Data;”

U.S. patent application Ser. No. 11/305,946 filed Dec. 19, 2005 entitled “Method, System, and Computer Program Product for the Evaluation of Glycemic Control in Diabetes from Self-Monitoring Data” (Publication No. 2006/0094947);

PCT International Application Serial No. PCT/US2003/025053, filed Aug. 8, 2003, entitled “Method, System, and Computer Program Product for the Processing of Self-Monitoring Blood Glucose (SMBG) Data to Enhance Diabetic Self-Management;”

U.S. patent application Ser. No. 10/524,094 filed Feb. 9, 2005 entitled “Managing and Processing Self-Monitoring Blood Glucose” (Publication No. 2005/214892);

PCT International Application Serial No PCT/US2006/033724, filed Aug. 29, 2006, entitled “Method for Improvising Accuracy of Continuous Glucose Sensors and a Continuous Glucose Sensor Using the Same;”

PCT International Application No. PCT/US2007/000370, filed Jan. 5, 2007, entitled “Method, System and Computer Program Product for Evaluation of Blood Glucose Variability in Diabetes from Self-Monitoring Data;”

U.S. patent application Ser. No. 11/925,689, filed Oct. 26, 2007, entitled “For Method, System and Computer Program Product for Real-Time Detection of Sensitivity Decline in Analyte Sensors;”

PCT International Application No. PCT/US00/22886, filed Aug. 21, 2000, entitled “Method and Apparatus for Predicting the Risk of Hypoglycemia;”

U.S. Pat. No. 6,923,763 B1, issued Aug. 2, 2005, entitled “Method and Apparatus for Predicting the Risk of Hypoglycemia;” and

PCT International Patent Application No. PCT/US2007/082744, filed Oct. 26, 2007, entitled “For Method, System and Computer Program Product for Real-Time Detection of Sensitivity Decline in Analyte Sensors.”

### REFERENCES CITED

The following patents, applications and publications as listed below and throughout this document are hereby incorporated by reference in their entirety herein. Moreover, the devices, systems, compositions, and computer program products and methods of various embodiments of the present invention disclosed herein may utilize aspects disclosed in the following U.S. Patents, foreign patents, and publications.
1. Basu A., Dalla Man C., Toffolo G., Basu R., Cobelli C., Rizza A., Effect of Type 2 Diabetes on Meal Glucose Fluxes and Insulin Secretion. Diabetes. 53 (suppl. 2), A579, 2004.
2. Basu R, Breda E, Oberg A L, Powell C C, Dalla Man C, Basu A, Vittone J L, Klee G G, Arora P, Jensen M D, Toffolo G, Cobelli C, Rizza R A. Mechanisms of the age-associated deterioration in glucose tolerance: contribution of alterations in insulin secretion, action, and clearance. Diabetes. 52:1738-48, 2003
3. Bergman R N, Finegood D T, Ader M. Assessment of insulin sensitivity in vivo. Am J Physiol 236:E667-677, 1985
4. Bergman R N, Ider Y Z, Bowden C R, Cobelli C. Quantitative estimation of insulin sensitivity. Am J Physiol. 236: E667-E677, 1979
5. Bergman R N, Phillips L S, Cobelli C: Physiologic evaluation of factors controlling glucose tolerance in man: measurement of insulin sensitivity and beta-cell glucose sensitivity from the response to intravenous glucose. J Clin Invest 68:1456-1467, 1981
6. Bergman R N. The minimal model of glucose regulation: a biography. Advances in Experimental Medicine & Biology 537:1-19, 2003
7. Bergman R N. Zaccaro D J. Watanabe R M. Haffner S M. Saad M F. Norris J M. Wagenknecht L E. Hokanson J E. Rotter J I. Rich S S. Minimal model-based insulin sensitivity has greater heritability and a different genetic basis than homeostasis model assessment or fasting insulin. Diabetes 52:2168-74, 2003
8. Boden G, Chen X, Urbain J L. Evidence for a circadian rhythm of insulin sensitivity in patients with NIDDM caused by cyclic changes in hepatic glucose production. Diabetes 45:1044-1050, 1996
9. Breda E, Cavaghan M K, Toffolo G, Polonsky K S, and Cobelli C. Oral glucose tolerance test minimal model indexes of beta-cell function and insulin sensitivity. Diabetes 50:150-158, 2001
10. Caumo A. Bergman R N. Cobelli C. Insulin sensitivity from meal tolerance tests in normal subjects: a minimal model index. Journal of Clinical Endocrinology & Metabolism. 85:4396-402, 2000
11. Clausen J O. Borch-Johnsen K. Ibsen H. Bergman R N. Hougaard P. Winther K. Pedersen O. Insulin sensitivity index, acute insulin response, and glucose effectiveness in a population-based sample of 380 young healthy Caucasians. Analysis of the impact of gender, body fat, physical fitness, and life-style factors. Journal of Clinical Investigation 98: 1195-209, 1996
12. Cobelli C. Measurement of Insulin Sensitivity and β-cell Function from Intravenous and Oral Glucose Tolerance Tests: Necessity of Models. Presentation at UVA Diabetes Endocrine Research Center, Nov. 7, 2005.
13. Dalla Man C, Caumo A, and Cobelli C. The oral glucose minimal model: estimation of insulin sensitivity from a meal test. IEEE Trans Biomed Eng 49: 419-429, 2002
14. Dalla Man C, Caumo A, Basu R, Rizza R, Toffolo G, and Cobelli C. Minimal model estimation of glucose absorption and insulin sensitivity from oral test: validation with a tracer method. Am J Physiol Endocrinol Metab 287: E637-E643, 2004
15. Dalla Man C, Caumo A, Basu R, Rizza R, Toffolo G, and Cobelli C. Measurement of selective effect of insulin on glucose disposal from labeled glucose oral test minimal model. Am J Physiol Endocrinol Metab 289: E909-E914, 2005
16. Dalla Man C, Rizza R A, and Cobelli C. Meal Simulation of the Glucose-Insulin System IEEE Trans Biomed Eng, In press, 2006
17. Dawson D, Vincent M A, Barrett E J, Kaul S, Clark A, Leong-Poi H, Lindner J R. Capillary recruitment in skeletal muscle in response to exercise and hyperinsulinemia assessed with contrast-enhanced ultrasound. Am J Physiol Endocrinol Metab, 282: E714-E720, 2002
18. DeFronzo R A, Tobin J D and Andres R. Glucose clamp technique: A method for quantifying insulin secretion and resistance. Amer J Physiol 23: E214-223, 1979
19. Ferrannini E: Insulin resistance versus insulin deficiency in non-insulin dependent diabetes mellitus: problems and prospects. Endocr Rev 19: 477-490, 1998
20. Friedberg S J, Lam Y W F, Blum J J, Gregerman R I: Insulin absoption: a major factor in apparent insulin resistance and the control of type 2 diabetes mellitus. Metab Clin. Exp. 5:614-619, 2006
21. Flier J S: Syndromes of insulin resistance. In: Becker K L, ed. Principles and Practice of Endocrinology & Metabolism, 2nd ed. Philadelphia, Pa.: JB Lippincott Company; 1995: 1245-1259
22. Gerich J E: The genetic basis of type 2 diabetes mellitus: impaired insulin secretion versus impaired insulin sensitivity. Endocr Rev 19:491-503, 1998
23. Hanley A J G, D'Agostino R, Wagenknecht L E, Saad M F, Savage P J, Bergman R, Haffner S M. Increased Proinsulin Levels and Decreased Acute Insulin Response Independently Predict the Incidence of Type 2 Diabetes in the Insulin Resistance Atherosclerosis Study. Diabetes 51:1263-1270, 2002
24. Kahn C R. Insulin action, diabetogenes, and the cause of type II diabetes. Diabetes 43:1066-1084, 1994
25. Kahn S E, Prigcon R L, McCulloch D K, Boyko E J, Bergman R N, Schwartz M W, Neifing J L, Ward W K, Beard J C, Palmer J P, et al.: Quantification of the relationship between insulin sensitivity and β-cell function in human subjects. Evidence for a hyperbolic function. Diabetes 42:1663-1672, 1993.
26. Korytkowski M T, Berga Sl, Horwitz M J: Comparison of the minimal model and the hyperglycemic clamp for measuring insulin sensitivity and acute insulin response to glucose. Metabolism 44: 1121-1125, 1995
27. Kovatchev B P, Otto E, Cox D J, Gonder-Frederick L A, Clarke W L (2006). Evaluation of a New Measure of Blood Glucose Variability in Diabetes. Diabetes Care, 29: 2433-2438.
28. Lee A, Ader M, Bray G A, Bergman R N. Diurnal variation in glucose tolerance. Cyclic suppression of insulin action and insulin secretion in normal-weight, but not obese, subjects. Diabetes 41:742-749, 1992
29. Liefmann R. Endocrine imbalance in rheumatoid arthritis and rheumatoid spondylitis; hyperglycemia unresponsiveness, insulin resistance, increased gluconeogenesis and mesenchymal tissue degeneration; preliminary report. Acta Med Scand. 136: 226-32, 1949
30. Mikines K J, Sonne B, Farrell P A, Tronier B, and Galbo H. Effect of physical exercise on sensitivity and responsiveness to insulin in humans. Am J Physiol Endocrinol Metab 254: E248-E259, 1988
31. Ni T C. Ader M. Bergman R N. Reassessment of glucose effectiveness and insulin sensitivity from minimal model analysis: a theoretical evaluation of the single-compartment glucose distribution assumption. Diabetes 46:1813-21, 1997
32. Reaven G M: Pathophysiology of insulin resistance in human disease. Physiol Rev. 75: 473-86, 1995
33. Richter E A. Glucose utilization. In: Handbook of Physiology, edited by LB Rowell and JT Shepherd. New York, N.Y.: Oxford University Press, 1996, 912-951
34. Toffolo G, Breda E, Cavaghan M K, Ehrmann D A, Polonsky K S, and Cobelli C. Quantitative indexes of beta-cell function during graded up & down glucose infusion from C-peptide minimal models. Am J Physiol Endocrinol Metab 280: E2-E10, 2001
35. Toffolo G, Cefalu W T, Cobelli C: β-cell function during insulin-modified intravenous glucose tolerance test successfully assessed by the C-peptide minimal model. Metabolism 48:1162-1166, 1999
36. Toffolo G, DeGrandi F, Cobelli C: Estimation of β-cell sensitivity from intravenous glucose tolerance tests C-peptide data. Diabetes 44: 845-854, 1995
37. Wahren J. Quantitative aspects of blood flow and oxygen uptake in the human forearm during rhythmic exercise. Acta Physiologica Scandinavica 67:5-10, 1966
38. Wasserman D H, Geer R J, Rice D E, Bracy D, Flakoll P J, Brown L L, Hill J O, and Abumrad N N. Interaction of exercise and insulin action in humans. Am J Physiol Endocrinol Metab 260: E37-E45, 1991
39. Weyer C, Bogardus C, Mott D M, Pratley R E: The natural history of insulin secretory dysfunction and insulin resistance in the pathogenesis of type 2 diabetes mellitus. J Clin Invest 104:787-794, 1999.

The devices, systems, computer systems, computer methods, devices, methods and computer program products of various embodiments of the invention disclosed herein may utilize aspects disclosed in the following U.S. Patents, foreign patents, and publications and are hereby incorporated by reference herein in their entirety:
Diabetes Management System and Method For Controlling Blood Glucose, PCT Application No. PCT/US1999/022586, Worthington, et al., filed Sep. 28, 1999 (Publication WO 00/18293);
System for Determining Insulin Dose Using Carbohydrate to Insulin Ratio and Insulin Sensitivity Factor, U.S. Patent Application Pub. No. 2005/0192494 A1, Barry H. Ginsberg, published Sep. 1, 2005;
System and Method for Measuring and Predicting Insulin Dosing Rates, U.S. Patent Application Publication No. 2007/0078314, Grounsell, et al., published Apr. 5, 2007;
Insulin Bolus Recommendation System, U.S. Patent Application No. 2006/0047192, Hellwig, et al., published Mar. 2, 2006;
Determination for Determining Insulin Drug Dose Using Carbohydrate to Insulin Ratio and Insulin Sensitivity Factor, U.S. Patent Application Pub. No. 2004/0197846, Hockersmith, et al., published Oct. 7, 2004;
System and Method for Portable Personal Diabetic Management, U.S. Patent Application Pub. No. 2003/0040821, Case, Christopher, published Feb. 27, 2003.
Diabetes Management System, U.S. Patent Application Pub. No. 2003/0028089, Galley, et al., published Feb. 6, 2003; and
Use of Targeted Glycemic Profiles in the Calibration of a NonInvasive Blood Glucose Monitor, PCT/US2001/047751 to Hockersmith, et al., published Sep. 19, 2002. (Publication WO 02/057740 A2).

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the present invention may have a variety of sizes, contours, shapes, compositions and materials as desired or required.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or socking priority hereto, then any such conflicting information in such incorporated by reference material is specifically not incorporated by reference herein.

What is claimed is:

1. A processor implemented method of measuring blood glucose variability in a diabetic human, combining blood glucose variability with a personal score computed from personal parameters of said diabetic human to compute insulin sensitivity (SI) of said diabetic human, and applying the SI of said diabetic human to manage at least one component of diabetes management of said diabetic human, comprising:
computing, by a processor, an estimate of the diabetic human's SI from routine self-monitoring blood glucose (SMBG) data; and
using said SI to derive said at least one component of said diabetes management for said diabetic human, selected from the group consisting of:
a carbohydrate ratio used to estimate the amount of insulin needed to compensate for an upcoming meal, or
a correction factor used to adjust the insulin amount so that a target glucose level can be reached, or
both said carbohydrate ratio and said correction factor,
wherein computing an estimate of SI comprises:
processing, by a processor, said SMBG data to determine blood glucose variability in said diabetic human; and
combining, by a processor, said determined blood glucose variability with a personal score computed for said diabetic human from parameters including the diabetic human's age, body mass index, insulin units per kilogram weight and the duration of the diabetes in the diabetic human.

2. The processor implemented method of claim 1 wherein said determined blood glucose variability comprises the processor computing average daily risk range (ADRR) for said diabetic human.

3. The processor implemented method of claim 1 wherein said combining of said determined blood glucose variability comprises linear type combining by said processor.

4. The processor implemented method of claim 1 wherein said personal score is referred to as SCORE, and whereby determining said SCORE comprises the following processor implemented algorithm:
wherein SCORE=0, and
if the age of said user is greater than 40 then said SCORE=SCORE+1,
if said duration is greater than 20 then said SCORE=SCORE+1,
if said BMI is less than 30 then said SCORE=SCORE+1, and
if said insulin units per kilogram is less than 0.5 then said SCORE=SCORE+1

5. The processor implemented method of claim 1 wherein said carbohydrate ratio is computed by the processor performing the steps of:
determining total insulin dependent glucose clearance (TIDGC) [mg·kg⁻¹] as:
TIDGC=∫S_{I}·(I−I_{b})=S_{I}∫(I−I_{b});
wherein I_{b} stands for basal insulin,
wherein S_{I} stands for insulin sensitivity,
calculating the following:
$$\dot{I} = - N \cdot I + \frac{{rate}_{bolus} + {rate}_{basal}}{V}$$
$$\begin{matrix}\int ( I - {I}_{b} ) = \int \frac{{rate}_{bolus}}{N \cdot V} - \frac{1}{N} \dot{I} \\ = \frac{1}{N \cdot V} \int {rate}_{bolus} - 0 \\ = \frac{bolus}{N \cdot V} \\ = \frac{bolus}{CL}\end{matrix}$$
wherein N is a time constant of insulin diffusion,
wherein V is a volume of insulin diffusion,
calculating the following:
$$TIDGC = {S}_{I} \frac{bolus [ mU ]}{CL}$$
wherein CL is a subject-specific parameter dependent on insulin clearance and insulin diffusion volume;
wherein CL is approximated using field-measurable subject characteristics as follows:
CL=^{-0.2+0.45·BSA-0.00287·age}
BSA=0.20247·Height[m]^{0.725}·W[kg]^{0.425}
where BSA stands for body surface are;
computing the total amount of glucose per kg ingested TGI using the formula:
$$TGI = \frac{1000 \cdot meal amount [ g ]}{weight [ kg ]}$$
computing the formula, wherein TGI is equated to TIDGC for an optimal bolus as follows:
$$TGI = TIDGC$$
$$1000 \frac{meal}{weight} = {S}_{I} \frac{bolus [ mU ]}{CL} and$$
$$\frac{bolus [ U ]}{meal [ g ]} = \frac{CL}{{S}_{I} \cdot weight [ kg ]}$$
$$and$$
$$said carb ratio = \frac{CL}{{S}_{I} \cdot weight [ kg ]} .$$

6. The processor implemented method of claim 1 where the processor computes said correction factor as follows:
$$TIDGC = \frac{( BG - {BG}_{target} ) Vol [ dl ]}{weight}$$
$${S}_{I} \frac{bolus [ mU ]}{CL} = \frac{Δ BG \cdot Vol [ dl ]}{weight} , and$$
$$\frac{bolus [ U ]}{Δ BG [ mg \text{/} dl ]} = 0.001 \times Vol [ dL ] \frac{CL}{{S}_{I} \cdot weight [ kg ]}$$
wherein ΔBG=(BG−BG_{target}), and Vol stands for glucose diffusion volume
calculating the formula:
correction factor=0.001×Vol[dI]×carbratio.

7. The processor implemented method of claim 1 wherein for rapid acting insulin situations said carbohydrate ratio said correction factor may be adjusted, whereby they are computed as follows:
CarbRatio_fast=CarbRatio/reach factor, and
CorrectionFactor_fast=CorrectionFactor/reach factor.

8. The processor implemented method of claim 7 wherein said reach factor is about 0.75.

9. A system for measuring blood glucose variability in a diabetic human, combining blood glucose variability with a personal score computed from personal parameters of said diabetic human to compute insulin sensitivity (SI) of said diabetic human, and applying the SI of said diabetic human to manage at least one component of diabetes management of said diabetic human, said system comprising:
an acquisition module acquiring a plurality of self-monitoring blood glucose (SMBG) data points representing blood glucose levels of said diabetic human, and
a processor programmed to estimate the SI from said SMBG data points and to derive at least one component of diabetes management using the estimated SI;
wherein said at least one component comprises:
a carbohydrate ratio used to estimate the amount of insulin needed to compensate for an upcoming meal, or
a correction factor used to adjust insulin amount so a target glucose level can be reached, or
both said carbohydrate ratio and said correction factor,
the processor programmed to estimate SI by processing the acquired SMBG data points to determine blood glucose variability, and to combine said determined blood glucose variability with a personal score computed from parameters including the diabetic human's age, body mass index, insulin units per kilogram weight and the duration of the diabetes in the diabetic human.

10. The system of claim 9 wherein said determined blood glucose variability comprises computing average daily risk range (ADRR).

11. The system of claim 9 wherein said combining of said determined blood glucose variability comprises linear type combining.

12. The system of claim 9 wherein said personal score is referred to as SCORE, and whereby determining said SCORE comprises the following computer algorithm:
wherein SCORE=0, and
if said age of user is greater than 40 then said SCORE=SCORE+1,
if said duration is greater than 20 then said SCORE=SCORE+1,
if said BMI less than 30 then said SCORE=SCORE+1, and
if said insulin units per kilogram is less than 0.5 then said SCORE=SCORE+1.

13. The system of claim 9 wherein said carbohydrate ratio is computed as follows:
determining total insulin dependent glucose clearance (TIDGC) [mg·kg⁻¹] as:
TIDGC=∫S_{I}·(I−I_{b})=S_{I}∫(I−I_{b});
wherein I_{b} stands for basal insulin,
wherein S_{I} stands for insulin sensitivity,
calculating the following:
$$\dot{I} = - N \cdot I + \frac{{rate}_{bolus} + {rate}_{basal}}{V}$$
$$\begin{matrix}\int ( I - {I}_{b} ) = \int \frac{{rate}_{bolus}}{N \cdot V} - \frac{1}{N} \dot{I} \\ = \frac{1}{N \cdot V} \int {rate}_{bolus} - 0 \\ = \frac{bolus}{N \cdot V} \\ = \frac{bolus}{CL}\end{matrix}$$
wherein N is a time constant of insulin diffusion
wherein V is a volume of insulin diffusion,
calculating the following:
$$TIDGC = {S}_{I} \frac{bolus [ mU ]}{CL}$$
wherein CL is a subject-specific parameter dependent on insulin clearance and insulin diffusion volume;
wherein CL is approximated using field-measurable subject characteristics as follows
CL=e^{-0.2+0.45·BSA-0.00287·age}
BSA=0.20247·Height[m]^{0.725}·W[kg]^{0.425}
where BSA stands for body surface area,
computing the formula, where TGI is equated to TIDGC for an optimal bolus, as follows:
$$TGI = TIDGC$$
$$1000 \frac{meal}{weight} = {S}_{I} \frac{bolus [ mU ]}{CL} and$$
$$\frac{bolus [ U ]}{meal [ g ]} = \frac{CL}{{S}_{I} \cdot weight [ kg ]}$$
$$and$$
$$said carb ratio = \frac{CL}{{S}_{I} \cdot weight [ kg ]} .$$

14. The system of claim 10 wherein said correction factor is computed as follows:
$$TIDGC = \frac{( BG - {BG}_{target} ) Vol [ dl ]}{weight}$$
$${S}_{I} \frac{bolus [ mU ]}{CL} = \frac{Δ BG \cdot Vol [ dl ]}{weight} , and$$
$$\frac{bolus [ U ]}{Δ BG [ mg \text{/} dl ]} = 0.001 \times Vol [ dL ] \frac{CL}{{S}_{I} \cdot weight [ kg ]}$$
wherein ΔBG=(BG−BG_{target}) and Vol stands for glucose diffusion volume
calculating the formula:
correction factor=0.001×Vol[dI]×carbratio.

15. The system of claim 9 wherein for rapid acting insulin situations said carbohydrate ratio and said correction factor may be adjusted, whereby they are computed as follows:
CarbRatio_fast=CarbRatio/reach factor, and
CorrectionFactor_fast=CorrectionFactor/reach factor.

16. The system of claim 15 wherein said reach factor is about 0.75.

17. A computer program product comprising a non-transitory computer readable medium having stored therein computer executable instructions for enabling at least one processor in a computer system to determine blood glucose variability of a diabetic human, combine blood glucose variability with a personal score computed from personal parameters of said diabetic human, and apply a computed insulin sensitivity (SI) of said diabetic human to manage at least one component of diabetes management of said diabetic human, said computer executable instructions comprising:
computing an estimate of the diabetic human's SI from routine self-monitoring blood glucose (SMBG) data,
using said SI to derive said at least one component of said diabetes management for said diabetic human, selected from the group consisting of:
a carbohydrate ratio used to estimate the amount of insulin needed to compensate for an upcoming meal, or
a correction factor used to adjust the insulin amount so that a target glucose level can be reached, or
both said carbohydrate ratio and said correction factor,
wherein computing an estimate of SI comprises:
processing said SMBG data to determine blood glucose variability in said diabetic human; and
combining said determined blood glucose variability with a personal score computed for said diabetic human from parameters including the diabetic human's age, body mass index, insulin units per kilogram weight and the duration of the diabetes in the diabetic human.
